(19) United States
(12) Patent Application Publication
(10) Pub. No.: US 2005/0201941 A1
(43) Pub. Date: Sep. 15, 2005

(54) Magnetic nanoparticle

(75) Inventors: Hui-Ju Cho, Lugang Township (TW); Sheng-Ming Shih, Taipei (TW); Yuh-Jiuan Lin, Taishan Township (TW); Hong-Dun Lin, Taipei City (TW); Kang-Ping Lin, Jungli City (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Mediatek Incorporation

(21) Appl. No.: 11/125,990

(22) Filed: May 10, 2005

Related U.S. Application Data
(62) Division of application No. 10/741,238, filed on Dec. 19, 2003.

(30) Foreign Application Priority Data
Jul. 31, 2003 (TW) 92120948

(51) Int. Cl. A61K 49/00

(52) U.S. Cl. 424/9.32

(57) ABSTRACT

A magnetic nanoparticle applicable in imaging, diagnosis, therapy and biomaterial separation. The magnetic nanoparticle is characterized as comprising at least an inner-transition element, represented as FeₓM^{a}ᵥZ_{y}, wherein M^{a} is an inner-transition element, Z is an element of the group VIa, x is greater or equal to 0, and both v and y are positive numbers. The magnetic nanoparticle may further comprise a shell to form a core-shell structure, wherein the shell is an inner-transition element M^{b} or the compound thereof.

### BACKGROUND OF THE INVENTION

1. Field of the Invention

The present invention relates to a magnetic nanoparticle applicable in imaging, diagnosis, therapy and biomaterial separation, and more particularly to a magnetic nanoparticle suitable for use as contrast agents in Magnetic Resonance Imaging.

2. Description of the Related Art

In the biotechnology field, a magnetic nanoparticle is applicable in imaging, diagnosis, therapy, biomaterial separation and so on. It is used, for example, in imaging as a contrast agent or a tracer to enhance the imaging contrast or to trace the presence of a certain disease. Furthermore, a magnetic nanoparticle is also applicable in drug delivery and cancer therapy.

Currently, a number of image analysis techniques such as Computer Topography (CT), Magnetic Resonance Imaging (MRI), and ultrasound (US) are applied in disease diagnosis. The popular analysis technique of computer topography employs an X-ray to image for example, a human body by X-ray diffraction of various tissues with various densities. In addition, a contrast agent may be added during analysis to enhance the contrast among different tissues or organs. However, the radiation of X-rays may bring undesired side effects, thus Magnetic Resonance Imaging (MRI) has been provided as an alternative analysis technique.

Magnetic resonance imaging is capable of showing selectively image several different characteristics of tissues. The level of tissue magnetization at specific signal recording times during the MR imaging cycle generally determines the brightness of a particular tissue in the MRI images. Contrast is produced when tissues do not have the same level of magnetization. There are three primary magnetic characteristics of tissue that are the source of image contrast. Two of these are associated with the longitudinal magnetization. They are proton density and T1, the longitudinal relaxation time. The third characteristic is associated with the transverse magnetization. It is T2, the transverse relaxation time.

Diagnosis of brain disorders has been markedly improved by using MRI, which can delineate detailed anatomic structures with excellent tissue contrast on T1, T2, and proton density-weighted images; however, the inherent tissue characteristics do not always produce adequate contrast for some clinical applications. The administer materials that will alter the magnetic characteristics within specific tissues or anatomical regions, and can disclose abnormal enhancement after intravenous administration of contrast agents due to brain-blood-barrier (BBB) disruption. Advanced MR imaging technique, which can detect in vivo physiological changes in human brain, such as water diffusion, blood volume and blood flow have been implemented in clinical MR scanners.

Certain materials are susceptible to magnetic field and become magnetized when located in field. The orbital electrons in the atom rather than magnetic properties of the nucleus determine the susceptibility of a material. Contrast agents used in MRI are generally based on susceptibility effects. Using dynamic susceptibility contrast technique takes the advantage of T2 signal changes during the first-pass of a bolus of contrast agents. Hemodynamic parameters can then be calculated in terms of cerebral blood volume (CBV), cerebral blood flow (CBF) and mean transit time (MTT) for diagnosis in clinical.

MRI provides a non-invasive diagnosis. An MRI with contrast agent enhancement increases sensitivity and specificity of imaging in many cases particularly when relaxation times among different tissues are similar.

MRI contrast agents can be classified differently according to their magnetic properties (paramagnetic, ferromagnetic or superparamagnetic). However, current commercial MRI contrast agents employing magnetic nanoparticles have poor specificity and their contrast enhancement could be improved.

### SUMMARY OF THE INVENTION

Accordingly, an object of the present invention is to provide a magnetic nanoparticle, applicable in imaging, diagnosis, therapy, biomaterial separation, thereby furthering development of its application as an MRI contrast agent.

Therefore, by utilizing a magnetic nanoparticle with an inner-transition element or forming an outer shell of an inner-transition element or its compound around the magnetic nanoparticle, the invention provides a magnetic nanoparticle. The magnetic nanoparticle can be selectively modified by at least one molecule (such as liposome, polymer, aliphatic compound or aromatic compound), or further react with at least one substance having specificity (such as an antibody, protein, peptide, enzyme, carbohydrate, glycoprotein, nucleotide or lipid) to form a contrast agent or tracer with specificity. Furthermore, the magnetic nanoparticle having specificity can perform a specific therapy such as killing cancer cells without harming healthy cells after entering the patient by heat transferred from the external magnetic field.

According to the invention, the provided magnetic nanoparticle applicable in imaging, diagnosis, therapy and biomaterial separation is represented as FeₓM^{a}ᵥZ_{y}, wherein M^{a} is an inner-transition element, Z is an element of the group VIa, x is greater than or equal to 0, while v and y are positive numbers.

According to the invention, the provided magnetic nanoparticle may further have a core-shell structure as shown in FIG. 1, in which the core 1-A is represented as FeₓM^{a}ᵥZ_{y} while the shell 1-B is made of an inner-transition element M^{b} or the compound thereof. Similarly, M^{a} is an inner-transition element, Z is an element of the group VIa, x is greater than or equal to 0, while v and y are positive numbers. M^{a} and M^{b} may be the same or different elements.

According to the invention, the inner-transition element M^{a} may be selected from the lanthanides or the actinides, and the element Z is, for example, oxygen or sulfur.

According to the invention, the magnetic nanoparticle can be further modified by at least one molecule, such as a liposome, polymer, aliphatic compound, aromatic compound or combinations thereof.

The modified magnetic nanoparticle may further react with at least one substance having specificity, such as an antibody, a protein, a peptide, an enzyme, a carbohydrate, a glycoprotein, a nucleotide or a lipid. In addition, the substances with specificity may directly react with the unmodified magnetic nanoparticle to give specificity thereto.

### DESCRIPTION OF THE DRAWINGS

The present invention can be more fully understood by reading the subsequent detailed description and examples with references made to the accompanying drawings, wherein:

FIG. 1 illustrates the core-shell structure of a magnetic nanoparticle of the invention;

FIGS. 2a-2d show the magnetic nanoparticles in the embodiment by Transmission Electron Microscope (TEM) observation;

FIG. 3 shows the X-ray diffraction (XRD) analysis of the magnetic nanoparticles in the embodiment;

FIG. 4 shows the Inductively Coupled Plasma—Atomic Emission Spectrometry (ICP-AES) analysis of the magnetic nanoparticles in the embodiment;

FIG. 5 shows the Super-conducting Quantum Interference Device (SQUID) analysis of the magnetic nanoparticles in the embodiment; and

FIG. 6 shows the Magnetic Resonance Imaging (MRI) analysis of the magnetic nanoparticles in the embodiment.

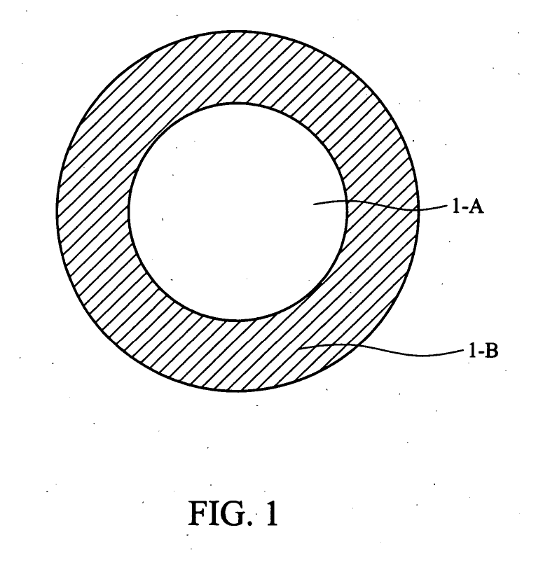

### DETAILED DESCRIPTION OF THE INVENTION

### Embodiment

In the embodiment, a magnetic nanoparticle of iron oxide comprising an inner-transition element of Gadolinium is given as an example, while the inner-transition element of the invention is not limited to this, for example, the inner-transition element can be any of the lanthanides or the actinides, and the compound of the inner-transition element can be an oxide, sulfide, selenide, telluride, or polonide of the inner-transition element. Also, the amount of the inner-transition element in the magnetic nanoparticle is not limited.

Preparation of Gd-Including Iron Oxide Nanoparticles

In the embodiment, Gd-including iron oxide nanoparticles were utilized as an MRI contrast agent.

First, a reaction flask was charged with FeCl₂ powders (0.0069 moles), FeCl₃ powders (0.0138 moles) and deionized water (30 ml). FeCl₃ powders were replaced by GdCl₃ in various ratios in other examples. NaOH with a concentration of 5M was added to control the pH value of the mixture. The mixture was subjected to continuous stirring during the reaction till the mixture became basic solution (the pH value approached about 11.5). Afterward, the temperature of the mixture was raised to and remained at 65° C. for 10 minutes. After black precipitates were formed, they were washed by deionized water and adjusted to acidic state by glacial acetic acid. Finally, H₂O₂ (10 vol %) was gradually added until the end of the gaseous reaction, and was followed by a deionized water wash.

Characterization of Gd-Including Iron Oxide Nanoparticles

1. Transmission Electron Microscope (TEM)

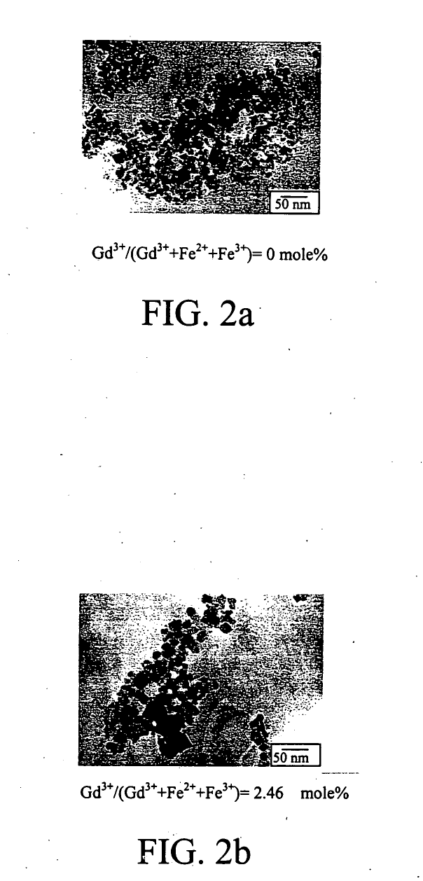

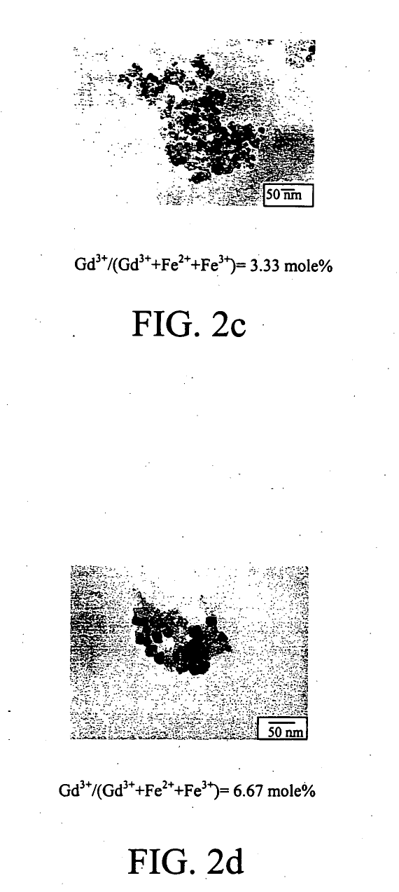

The magnetic nanoparticles were then observed by TEM (JOEL, 100CX II). FIGS. 2a-2d respectively show the magnetic nanoparticles with an initial Gd³⁺/(Gd³⁺+Fe²⁺+Fe³⁺) mixing ratio of 0, 2.46, 3.33 and 6.67 mol %. In these cases, their average diameters are about 8.2±1.6 nm, 14.6±2.7 nm, 19.6±3.2 nm and 22.1±3.5 nm, respectively.

2. X-Ray Diffraction (XRD)

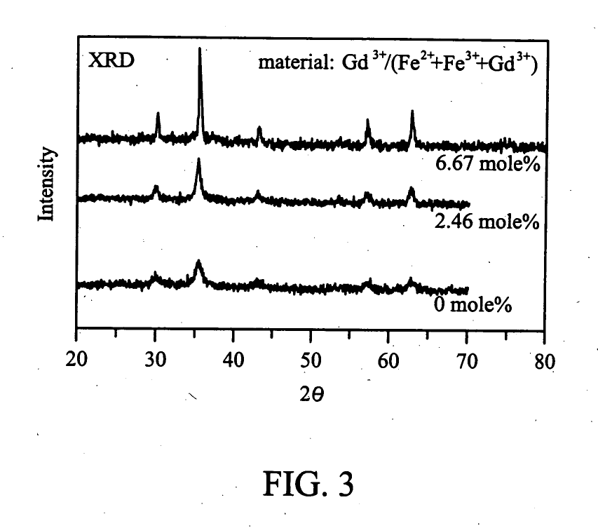

FIG. 3 shows the XRD analysis of the magnetic nanoparticles in the embodiment, further proving that the magnetic nanoparticles are iron oxide nanoparticles.

3. Inductively Coupled Plasma—Atomic Emission Spectrometry (ICP-AES)

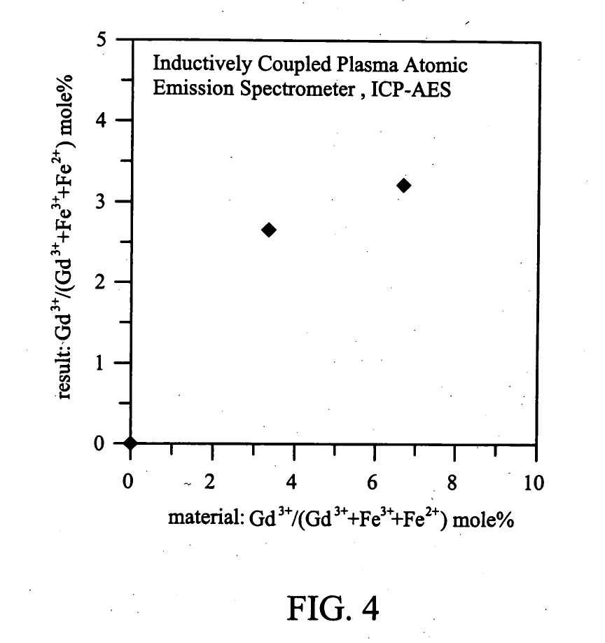

FIG. 4 shows the ICP-AES analysis of the magnetic nanoparticles in the embodiment. The magnetic nanoparticles with an initial Gd³⁺/(Gd³⁺+Fe²⁺+Fe³⁺) mixing ratio of 0 mol %, 3.33 mol % or 6.67 mol % have a final Gd³⁺/(Gd³⁺+Fe²⁺+Fe³⁺) ratio in the nanoparticles of 0 mol %, 2.65 mol % or 3.20 mol %.

4. Super-Conducting Quantum Interference Device (SQUID)

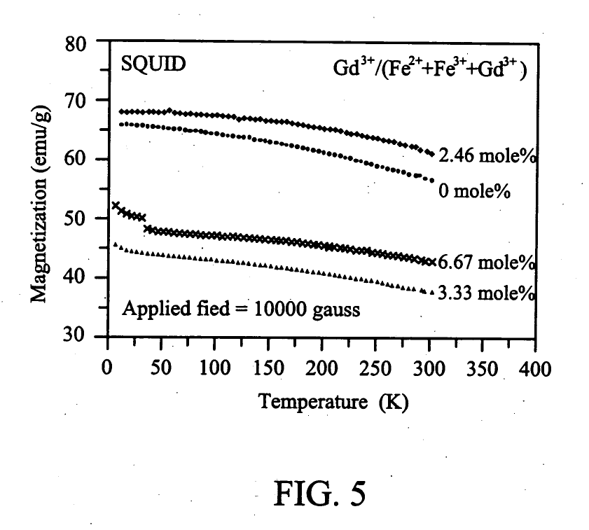

FIG. 5 shows the SQUID analysis of the magnetic nanoparticles in the embodiment. The results indicate a 3-8% increased magnetization of the magnetic nanoparticles having 2.46 mol % of GdCl₃ added.

5. Magnetic Resonance Imaging (MRI)

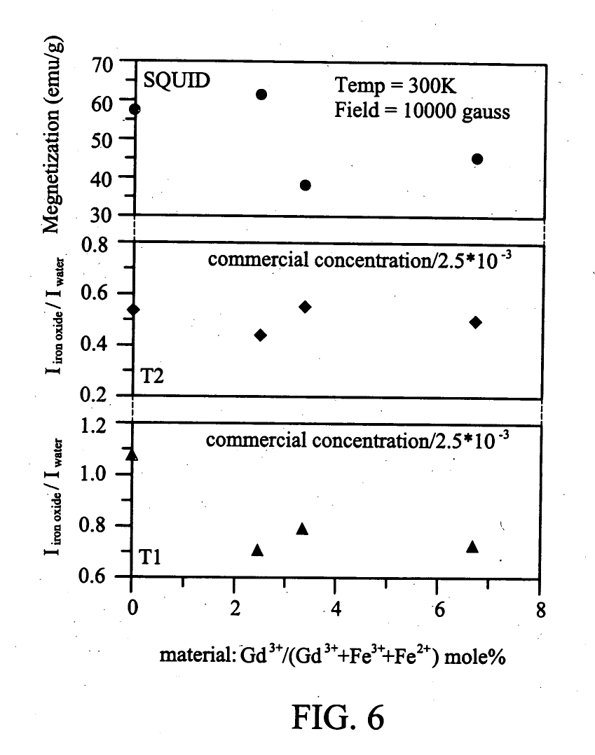

After clinically injecting a contrast agent, the concentration of the contrast agent is diluted by blood or body fluid, so the effective concentration is less than the concentration of the commercial contrast agent. Therefore, the provided magnetic nanoparticles were prepared as a contrast agent having a concentration 2.5×10⁻³ times that of a commercial MRI iron oxide contrast agent. FIG. 6 shows the MRI analysis using the magnetic nanoparticles as contrast agent. The longitudinal coordinates represent the signal intensity ratios of the oxides and water molecules. The greater the coordinates deviates from 1, the better the contrast enhancement is. As shown in FIG. 6, all of the four kinds of magnetic nanoparticles with various GdCl₃ additive ratios exhibited contrast-enhancing capability. Especially, the iron oxide nanoparticles having 2.46 mol % additive GdCl₃ increased the contrast 18% more than that having non additive GdCl₃ under T₂-weignted conditions.

Accordingly, the Gd-including iron oxide nanoparticles enhance the contrast effectively and provide a clearer MRI image. Furthermore, the provided Gd-including iron oxide nanoparticles may be selectively modified by a molecule such as a liposome, polymer, aliphatic compound, or aromatic compound. The modified magnetic nanoparticle may further react with a substance having specificity, such as an antibody, a protein, a peptide, an enzyme, a carbohydrate, a glycoprotein, a nucleotide or a lipid to form a contrast agent having specificity.

The foregoing description has been presented for purposes of illustration and description. Obvious modifications or variations are possible in light of the above teaching. The embodiment was chosen and described to provide the best illustration of the principles of this invention and its practical application to thereby enable those skilled in the art to utilize the invention in various embodiments and with various modifications as are suited to the particular use contemplated. All such modifications and variations are within the scope of the present invention as determined by the appended claims when interpreted in accordance with the breadth to which they are fairly, legally, and equitably entitled.

1-19. (canceled)

20. A magnetic nanoparticle comprising:
a core represented as FeₓM^{a}ᵥZ_{y}, wherein M^{a} is an inner-transition element, Z is an element of the group VIa, x is greater or equal to 0, and v, y are positive numbers; and
a shell of an inner-transition element M^{b} or the compound thereof.

21. The magnetic nanoparticle as claimed in claim 20, wherein the inner-transition elements M^{a} and M^{b} are selected from the lanthanides or the actinides.

22. The magnetic nanoparticle as claimed in claim 20, wherein the inner-transition elements M^{a} and M^{b} are the same element.

23. The magnetic nanoparticle as claimed in claim 20, wherein the inner-transition elements M^{a} and M^{b} are different elements.

24. The magnetic nanoparticle as claimed in claim 20, wherein the element Z is oxygen or sulfur.

25. The magnetic nanoparticle as claimed in claim 20, wherein the compound of the inner-transition element M^{b} is a complex.

26. The magnetic nanoparticle as claimed in claim 20, wherein the magnetic nanoparticle is applicable in imaging, diagnosis, therapy and biomaterial separation.

27. The magnetic nanoparticle as claimed in claim 20, further modified by at least one molecule.

28. The magnetic nanoparticle as claimed in claim 27, wherein the molecule is a liposome, polymer, aliphatic compound, aromatic compound or combinations thereof.

29. The magnetic nanoparticle as claimed in claim 20, wherein the magnetic nanoparticle further reacts with at least one substance having specificity.

30. The magnetic nanoparticle as claimed in claim 29, wherein the substance having specificity is an antibody, a protein, a peptide, an enzyme, a carbohydrate, a glycoprotein, a nucleotide or a lipid.

31. The magnetic nanoparticle as claimed in claim 27, wherein the magnetic nanoparticle further reacts with at least one substance having specificity.

32. The magnetic nanoparticle as claimed in claim 31, wherein the substance having specificity is an antibody, a protein, a peptide, an enzyme, a carbohydrate, a glycoprotein, a nucleotide or a lipid.

33. A magnetic nanoparticle applicable in imaging, diagnosis, therapy and biomaterial separation, comprising:
a core represented as FeₓM^{a}ᵥZ_{y}, wherein M^{a} is an inner-transition element, Z is an element of the group VIa, x is greater or equal to 0, and v, y are positive numbers; and
a shell of an inner-transition element M^{b} or the compound thereof.

34. The magnetic nanoparticle as claimed in claim 33, wherein the inner-transition elements M^{a} and M^{b} are selected from the lanthanides or the actinides.

35. The magnetic nanoparticle as claimed in claim 33, wherein the inner-transition elements M^{a} and M^{b} are the same element.

36. The magnetic nanoparticle as claimed in claim 33, wherein the inner-transition elements M^{a} and M^{b} are different elements.

37. The magnetic nanoparticle as claimed in claim 33, wherein the element Z is oxygen or sulfur.

38. The magnetic nanoparticle as claimed in claim 33, wherein the compound of the inner-transition element M^{b} is a complex.

39. The magnetic nanoparticle as claimed in claim 33, further modified by at least one molecule.

40. The magnetic nanoparticle as claimed in claim 39, wherein the molecule is a liposome, polymer, aliphatic compound, aromatic compound or combinations thereof.

41. The magnetic nanoparticle as claimed in claim 33, wherein the magnetic nanoparticle further reacts with at least one substance having specificity.

42. The magnetic nanoparticle as claimed in claim 41, wherein the substance having specificity is an antibody, a protein, a peptide, an enzyme, a carbohydrate, a glycoprotein, a nucleotide or a lipid.

43. The magnetic nanoparticle as claimed in claim 39, wherein the magnetic nanoparticle further reacts with at least one substance having specificity.

44. The magnetic nanoparticle as claimed in claim 43, wherein the substance having specificity is an antibody, a protein, a peptide, an enzyme, a carbohydrate, a glycoprotein, a nucleotide or a lipid.
